(19) United States
(12) Patent Application Publication
(10) Pub. No.: US 2026/0166326 A1
(43) Pub. Date: Jun. 18, 2026

(54) EXTERNAL LEAD CONNECTOR WITH LEAD MOVEMENT AND METHODS OF MAKING AND USING

(71) Applicant: Boston Scientific Neuromodulation Corporation, Valencia, CA (US)

(72) Inventor: Thomas Paul Lopez, Sunland, CA (US)

(21) Appl. No.: 19/420,301

(22) Filed: Dec. 15, 2025

Related U.S. Application Data
(60) Provisional application No. 63/735,238, filed on Dec. 17, 2024.

(51) Int. Cl. A61N 1/375; A61N 1/372; H01R 24/58

(52) U.S. Cl. CPC A61N 1/3752; A61N 1/37241; H01R 24/58; H01R 2201/12

(57) ABSTRACT

A device for electrically coupling a lead to an external trial stimulator includes a lead connector that has a housing defining a lead aperture and a sliding aperture, connector contacts, a slide frame, a button coupled to the slide frame and configured to move along the sliding aperture, and a bracket engaging the slide frame and defining a lead passage configured to receive a portion of the lead. The bracket includes upper flanges and lower flanges alternating along the lead passage. At least one of the slide frame or bracket defines a slanted slot and at least one of the slide frame or bracket includes a pin disposed within the slanted slot of the other of the slide frame or bracket. Moving the button moves the bracket between a loading position for receiving the lead and an engagement position, in which the lead engages the connector contacts.

### CROSS-REFERENCE TO RELATED APPLICATIONS

This application claims the benefit under 35 U.S.C. § 119 (e) of U.S. Provisional Patent Application Ser. No. 63/735,238, filed Dec. 17, 2024, which is incorporated herein by reference.

### FIELD

The present invention is directed to the area of implantable electrical stimulation systems and methods of making and using the systems. The present invention is also directed to an external lead connector for use with an implantable electrical stimulation system and an external trial stimulator, as well as methods of making and using the external lead connector.

### BACKGROUND

Implantable electrical stimulation systems have proven therapeutic in a variety of diseases and disorders. For example, spinal cord stimulation systems have been used as a therapeutic modality for the treatment of chronic pain syndromes. Functional electrical stimulation systems have been applied to restore some functionality to paralyzed extremities in spinal cord injury patients. Stimulation of the brain, such as deep brain stimulation, can be used to treat a variety of diseases or disorders. Peripheral nerve stimulation has been used to treat chronic pain syndrome and incontinence, with a number of other applications under investigation.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One aspect is a device for electrically coupling a lead to an external trial stimulator. The device includes a lead connector configured for electrically coupling the lead to the trial stimulator, the lead connector configured to receive a proximal end portion of the lead. The lead connector includes a housing defining a lead aperture in an end of the housing and a sliding aperture in a surface of the housing, a plurality of connector contacts electrically coupleable to the external trial stimulator, a slide frame, a button coupled to the slide frame and configured to move along the sliding aperture, and a bracket engaging the slide frame and defining a lead passage configured to receive the proximal end portion of the lead, the bracket including a plurality of upper flanges and a plurality of lower flanges, wherein the upper flanges and lower flanges alternate along the lead passage, wherein at least one of the slide frame or the bracket define at least one slanted slot and at least one of the slide frame or the bracket includes at least one pin disposed within the at least one slanted slot of the other of the slide frame or the bracket, wherein moving the button along the sliding aperture moves the bracket between a loading position, in which the proximal end portion of the lead is receivable through the lead aperture into the lead passage, and an engagement position, in which the proximal end portion of the lead, when received in the lead passage, engages the connector contacts.

In at least some aspects, the lead connector is configured to receive the proximal end portions of a single lead or of a plurality of leads, wherein the lead connector includes a plurality of the lead apertures and a plurality of the lead brackets, wherein each of the lead brackets defines a lead passage aligned with a different one of the lead apertures. In at least some aspects, the lead connector includes a plurality of the buttons and a plurality of the slide frames, wherein each slide frame is coupled to a different one of the buttons and engages a different one of the brackets. In at least some aspects, the plurality of the lead brackets each engage the slide frame.

In at least some aspects, the connector contacts each include an “M” pin. In at least some aspects, the lead connector is configured for direct attachment to the external trial stimulator. In at least some aspects, the device further includes an elongated body having a first end and a second end, wherein the first end is coupled or coupleable to the external trial stimulator and the second end is coupled or coupleable to the lead connector. In at least some aspects, at least one of the at least one slanted slot defines a curved path. In at least some aspects, at least one of the at least one slanted slot defines a straight path.

In at least some aspects, the housing further includes a stylet aperture intersecting the lead aperture and extending along a side of the housing. In at least some aspects, the upper and lower flanges define a gap configured to allow a stylet extending from the lead and passing the stylet aperture to enter the lead passage.

In at least some aspects, the bracket further includes an end stop configured to prevent or resist further insertion of the proximal end portion of the electrical stimulation lead into the lead connector. In at least some aspects, each of the upper flanges and lower flanges has a “J” or “U” shape.

Another aspect is a trial stimulation system that includes an external trial stimulator and any of the devices described above coupleable, or coupled, to the external trial stimulator.

In at least some aspects, the trial stimulation system further includes an elongated body coupled or coupleable to the lead connector and the external trial stimulator. In at least some aspects, the lead connector is configured for direct attachment to the external trial stimulator.

In at least some aspects, the trial stimulation system further includes the lead coupleable to the lead connector. In at least some aspects, the trial stimulation system further includes a stylet configured for insertion into a stylet lumen of the lead. In at least some aspects, the trial stimulation system further includes a lead extension coupleable to the lead and the lead connector.

A further aspect is an insertion kit that includes any of the devices described above and at least one electrical stimulation lead. Each electrical stimulation lead has a distal end portion and a proximal end portion and includes a plurality of electrodes disposed along the distal end portion of the electrical stimulation lead, a plurality of terminals disposed along the proximal end portion of the electrical stimulation lead, and a plurality of conductors coupling the electrodes to the terminals, wherein the proximal end portion of the electrical stimulation lead is insertable into the lead connector.

Yet another aspect is a method for performing a trial stimulation on a patient that includes providing any of the devices described above; inserting a proximal end portion of the lead into the lead connector while the bracket is in the loading position; and moving the bracket to the engagement position to engage terminals along the proximal end portion of the lead with the connector contacts of the lead connector.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a percutaneous lead electrically coupled to a control module, according to the invention;

FIG. 2A is a schematic view of one embodiment of the control module of FIG. 1 configured and arranged to electrically couple to an elongated device, according to the invention;

FIG. 2B is a schematic view of one embodiment of a lead extension configured and arranged to electrically couple the elongated device to the control module of FIG. 1, according to the invention;

FIG. 3 is a schematic view of one embodiment of a stylet for insertion into a proximal end portion of an electrical stimulation lead;

FIG. 4 is a schematic illustration of components of one embodiment of a trial stimulation system, according to the invention;

FIG. 5A is a schematic perspective view of one embodiment of a lead connector as part of an operating room cable, according to the invention;

FIG. 5B is a schematic perspective cross-sectional view of the lead connector of FIG. 5A, according to the invention;

FIG. 5C is another schematic perspective cross-sectional view of the lead connector of FIG. 5A, according to the invention;

FIG. 6A is a schematic perspective cross-sectional view of a portion of another embodiment of a lead connector with the bracket in a loading position, according to the invention;

FIG. 6B is a schematic perspective cross-sectional view of the portion of the lead connector of FIG. 6A with the bracket in an engagement position, according to the invention;

FIG. 6C is a schematic perspective cross-sectional view of a bracket of the lead connector of FIG. 6A, according to the invention;

FIG. 6D is another schematic perspective cross-sectional view of the portion of the lead connector of FIG. 6A with the bracket in the loading position, according to the invention;

FIG. 6E is another schematic perspective cross-sectional view of the portion of the lead connector of FIG. 6A with the bracket in the engagement position, according to the invention;

FIG. 7A is a schematic view of one embodiment of a lead connector directly attached to an external trial stimulator;

FIG. 7B is a schematic view of one embodiment of a lead connector coupled to an external trial stimulator by a separate operating room cable; and

FIG. 8 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to an external lead connector for use with an implantable electrical stimulation system, as well as methods of making and using the external lead connector.

Suitable implantable electrical stimulation systems include, but are not limited to, a least one lead with one or more electrodes disposed along a distal end of the lead and one or more terminals disposed along the one or more proximal end portions of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 8,175,710; 8,224,450; 8,271,094; 8,295,944; 8,364,278; and 8,391,985; U.S. Patent Application Publications Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, as well as the other references cited herein, all of which are incorporated by reference in their entireties.

An electrical stimulation lead will be used as an example herein but it will be understood that the lead connectors and operating room cables described below can also be used with optical stimulation or modulation leads and electrical/optical stimulation leads. Examples of optical stimulation or modulation systems or electrical/optical stimulation systems, which include one or more optical emitters in addition to, or as an alternative to, electrodes, are found in U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329, all of which are incorporated by reference in their entireties.

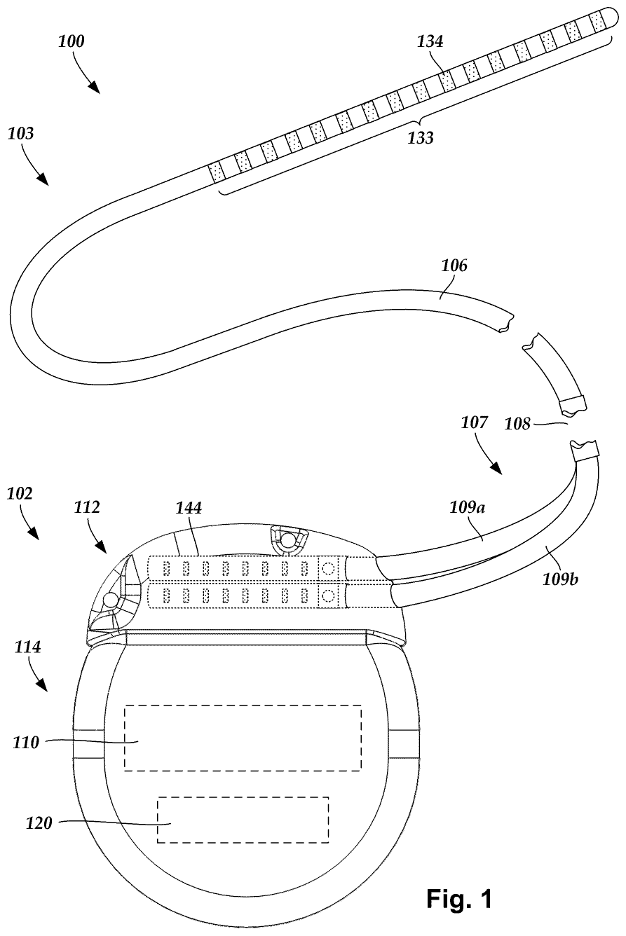

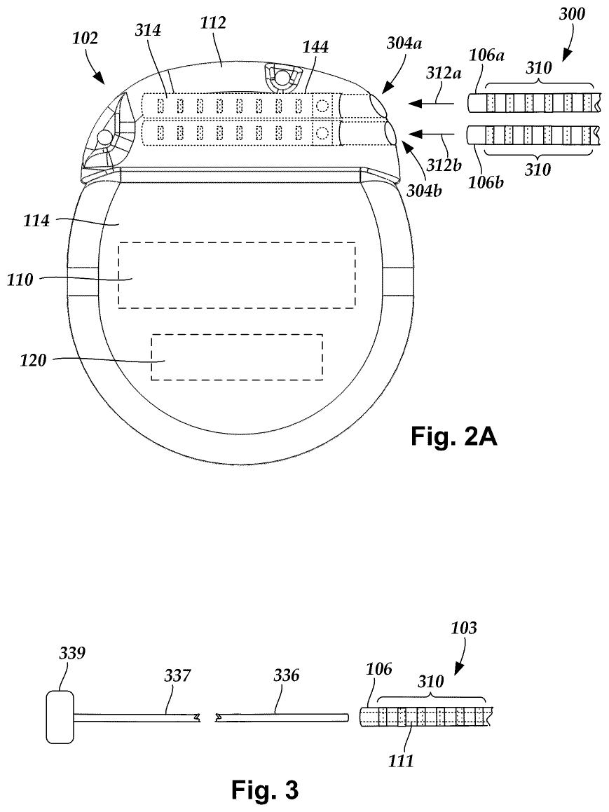

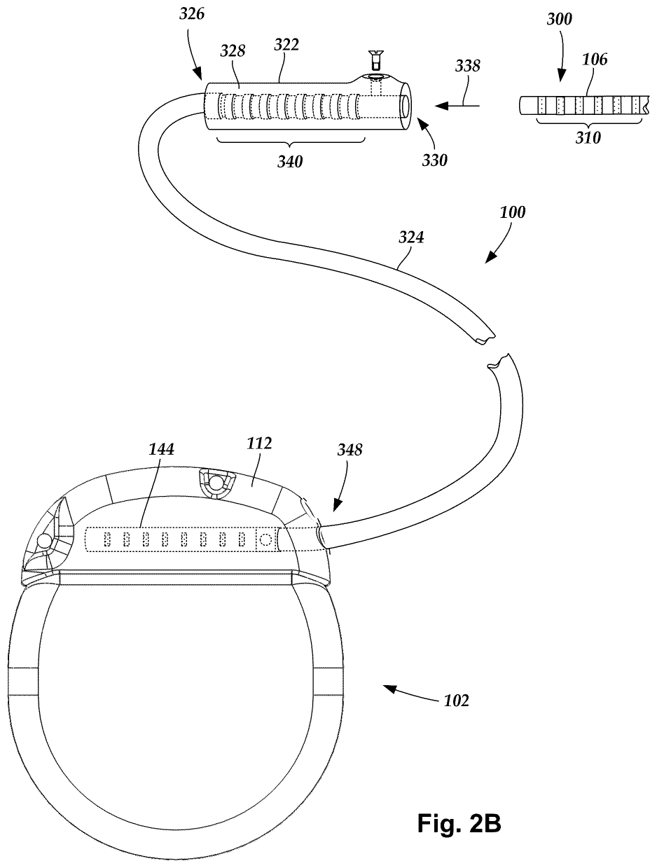

FIG. 1 illustrates schematically one embodiment of an electrical stimulation system 100. The electrical stimulation system includes a control module (e.g., a stimulator or pulse generator) 102 and a lead 103 coupleable to the control module 102. Any suitable number of leads can be coupled to a control module 102, such as one, two, three, four, or more leads. The lead 103 is a percutaneous lead, although it will be understood that any other type of lead can be used including, but not limited to, a paddle lead or a cuff lead. An array 133 of electrodes, such as electrode 134, is disposed along a distal end portion of the lead, and an array of terminals (e.g., 310 in FIG. 2A-2B) is disposed along a proximal end portion of the lead. In FIG. 1, the lead 103 is shown having one lead body 106 that is optionally split into two splitter tails 109a, 109b. It will be understood that the lead 103 can include any suitable number of lead bodies including, for example, one, two, three, four, five, six, seven, eight or more lead bodies 106. In at least some embodiments, the lead 103 is isodiametric along a longitudinal length of the lead body 106.

It will be understood that the electrical stimulation system can include more, fewer, or different components and can have a variety of different configurations including those configurations disclosed in the electrical stimulation system references cited herein.

The lead 103 can be coupled to the control module 102 in any suitable manner. In FIG. 1, the lead 103 is shown coupling directly to the control module 102. In at least some other embodiments, the lead 103 couples to the control module 102 via one or more intermediate devices. For example, in at least some embodiments one or more lead extensions 324 (FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 1, the electrical stimulation system 100 is shown having an optional splitter 107 configured and arranged for facilitating coupling of the lead 103 to the control module 102. The splitter 107 includes a splitter connector 108 configured to couple to a proximal end portion of the lead 103, and one or more splitter tails 109a and 109b configured and arranged to couple to the control module 102 (or another splitter, a lead extension, an adaptor, or the like).

The control module 102 typically includes a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including the one or more of the lead bodies 106 and the control module 102, may be implanted into the body of a patient. The electrical stimulation system can be used for a variety of applications including, but not limited to deep brain stimulation, neural stimulation, spinal cord stimulation, muscle stimulation, and the like.

Any suitable number of electrodes 134 can be disposed on the lead including, for example, four, five, six, seven, eight, nine, ten, eleven, twelve, fourteen, sixteen, twenty-four, thirty-two, or more electrodes 134. The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

The one or more lead bodies 106 can be made of a non-conductive, biocompatible material such as, for example, silicone, polyurethane, polyetheretherketone (“PEEK”), epoxy, and the like or any combination thereof. The one or more lead bodies 106 may be formed in the desired shape by any process including, for example, molding (including injection molding), casting, and the like. The non-conductive material typically extends from the distal ends of the one or more lead bodies 106 to the proximal end portion of each of the one or more lead bodies 106.

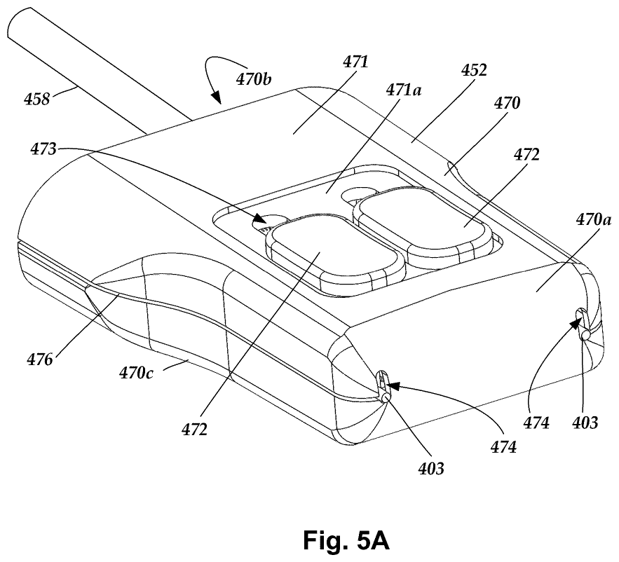

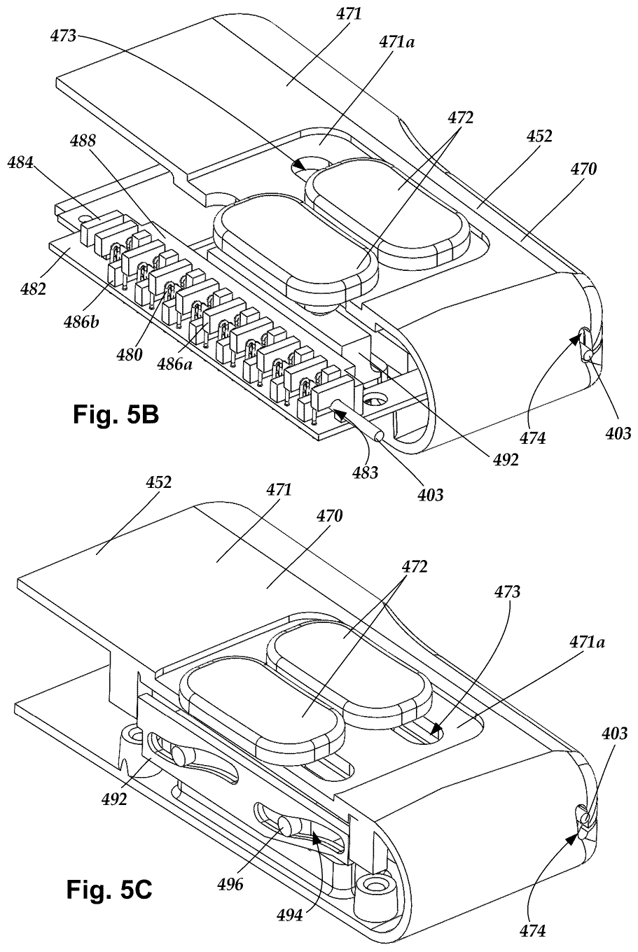

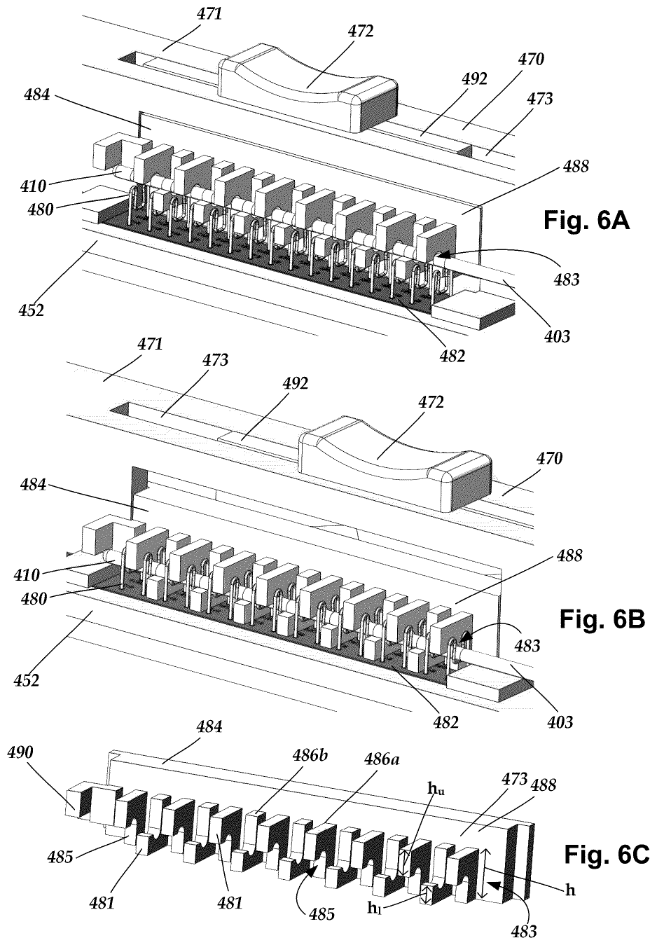

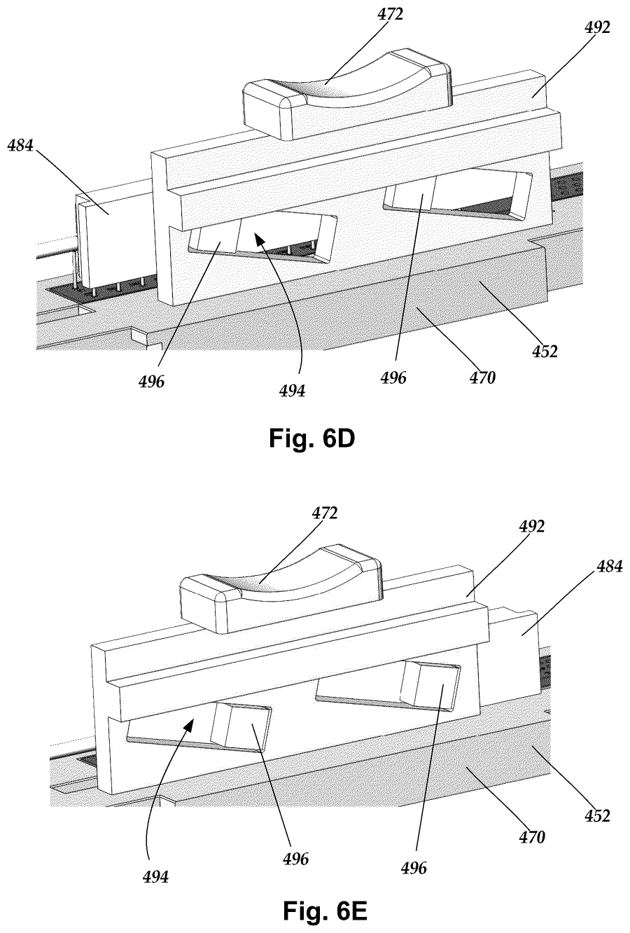

Terminals (e.g., 310 in FIGS. 2A-2B) are typically disposed along the proximal end portion of the one or more lead bodies 106 of the electrical stimulation system 100 (as well as any splitters, lead extensions, adaptors, or the like) for electrical connection to corresponding connector contacts (e.g., 314 in FIG. 2A or 480 in FIGS. 5B, 6A, 6B, and 6C). The connector contacts are disposed in connectors (e.g., 144 in FIGS. 1-2B; and 322 FIG. 2B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end portion of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 2A is a schematic side view of one embodiment of a proximal end portion of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 2B, an adaptor, or the like or combinations thereof), or a combination thereof.

The control module connector 144 defines at least one port into which a proximal end portion of the elongated device 300 can be inserted, as shown by directional arrows 312a and 312b. In FIG. 2A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end portion(s) of the elongated device(s) 300 to electrically couple the control module 102 to the electrodes 134 of the lead 103. Examples of connectors in control modules are found in, for example, U.S. Pat. Nos. 7,244,150 and 8,224,450, which are incorporated by reference.

FIG. 2B is a schematic side view of another embodiment of the electrical stimulation system 100. The electrical stimulation system 100 includes a lead extension 324 that is configured and arranged to couple one or more elongated devices 300 (e.g., one of the lead bodies 106, the splitter 107, an adaptor, another lead extension, or the like or combinations thereof) to the control module 102. In FIG. 2B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144, or to receive multiple elongated devices 300, or both.

A lead extension connector 322 is disposed on the lead extension 324. In FIG. 2B, the lead extension connector 322 is shown disposed at a distal end 326 of the lead extension 324. The lead extension connector 322 includes a connector housing 328. The connector housing 328 defines at least one port 330 into which terminals 310 of the elongated device 300 can be inserted, as shown by directional arrow 338. The connector housing 328 also includes a plurality of connector contacts, such as connector contacts 340. When the elongated device 300 is inserted into the port 330, the connector contacts 340 disposed in the connector housing 328 can be aligned with the terminals 310 of the elongated device 300 to electrically couple the lead extension 324 to the electrodes (134 of FIGS. 1 and 2) disposed along the lead (103 in FIGS. 1 and 2).

In at least some embodiments, the proximal end portion of the lead extension 324 is similarly configured and arranged as a proximal end portion of the lead 103 (or other elongated device 300). The lead extension 324 may include a plurality of electrically conductive wires (not shown) that electrically couple the connector contacts 340 to a proximal end portion 348 of the lead extension 324 that is opposite to the distal end 326. In at least some embodiments, the conductive wires disposed in the lead extension 324 can be electrically coupled to a plurality of terminals (not shown) disposed along the proximal end portion 348 of the lead extension 324. In at least some embodiments, the proximal end portion 348 of the lead extension 324 is configured and arranged for insertion into a connector disposed in another lead extension (or another intermediate device). In other embodiments (and as shown in FIG. 2B), the proximal end portion 348 of the lead extension 324 is configured and arranged for insertion into the control module connector 144.

FIG. 3 illustrates one embodiment of a stylet 336 that can be inserted into a lumen 111 of a lead body 106 of a lead 103 or other elongated body. The stylet 336 can stiffen the lead 103 to facilitate implantation of the lead into a patient. The stylet 336 can include, for example, a stylet shaft 337 and a handle 339.

During implantation of the lead into a patient it can be desirable to test the positioning or functionality of the electrodes within the patient prior to completion of the implantation. One way to test electrode positioning or functionality is to implant an electrode-including distal end portion of a lead (and, optionally, one or more lead extensions) into the patient. The proximal end portion of the lead (or lead extension) can then be electrically coupled to a trial stimulator that is disposed external to the patient to perform trial stimulations using the electrodes. Once it is determined that the electrodes are properly positioned and functioning within desired parameters, the trial stimulator can be decoupled from the proximal end portion of the lead (or lead extension) and replaced with an implantable control module, and the implantation can be completed.

In some embodiments, the trial stimulations can continue for two, four, six, eight, twelve, or more hours or for one, two, three, four, five or more days. In these instances, the patient may be in a hospital or other care facility. In some embodiments, the trial stimulations may continue for an extended period (e.g., 2-10 days or more) where the patient is sent home with the lead, cable, and trial stimulator to assess the effectiveness of the therapy to determine if a permanent implanted system will be effective in treating the medical condition. During the trial stimulations, the lead can be electrically coupled to the trial stimulator by electrically coupling the proximal end portion of the lead (or lead extension) to an operating room cable (“cable”) which, in turn, is electrically coupled to the trial stimulator. In some cases, when multiple leads are implanted into a patient, multiple leads (or lead extensions) may be coupled to the cable.

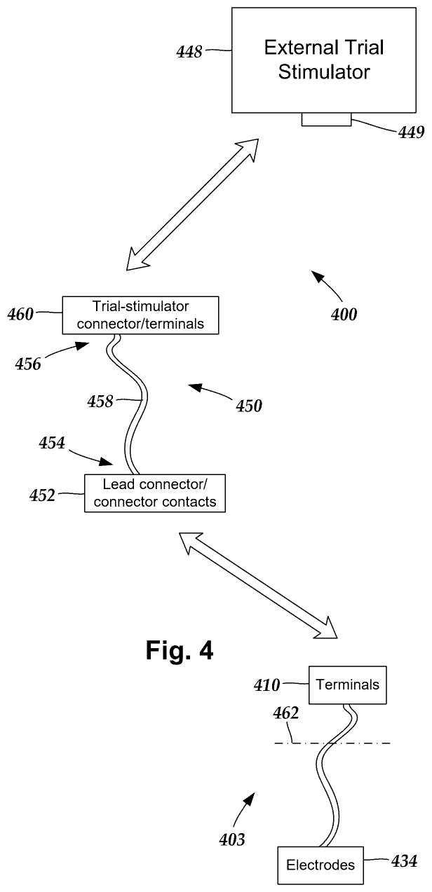

FIG. 4 is a schematic view of one embodiment of a trial stimulation arrangement 400 that includes a lead 403, a trial stimulator 448, and an operating room cable 450, that couples the lead 403 to the trial stimulator 448. The lead 403 includes an array of electrodes 434 and an array of terminals 410. The terminals 410 are configured and arranged to couple the electrodes 434 to the trial stimulator 448 when the operating room cable 450 is coupled to each of the lead 403 and the trial stimulator 448. Non-limiting examples of operating room cables include, but are not limited to, those disclosed in U.S. Pat. Nos. 7,539,542; 8,849,396; 9,101,775; 9,662,506; 10,130,806; 10,195,446; 10,639,485; and 11,045,656, all of which are incorporated herein by reference in their entireties. In at least some embodiments, the trial stimulator 448 can have any of the features or components of the control module 102, described herein.

During operation, the electrodes 434 are disposed internally to the patient, while the terminals 410 remain external to the patient, as shown in FIG. 4 by a line 462 schematically representing patient skin. Optionally, the trial stimulation arrangement 400 includes one or more additional devices (e.g., a lead extension, an operating room cable extension, a splitter, an adaptor, or the like or any combination thereof). For example, the trial stimulation arrangement can include a lead extension which is coupleable to, and between, the lead 403 and the operating room cable 450.

The operating room cable 450 includes an elongated body 458 (e.g., cord or cable) having a first end portion 454 and an opposing second end portion 456, a lead connector 452 with connector contacts, and an optional trial stimulator connector 460 optionally with terminals (a trial stimulator connector and terminals are not needed if the operating room cable is permanently wired, or otherwise permanently attached, to the trial stimulator). Conductors (not shown) extend from the connector contacts of the lead connector to the terminals of the trial stimulator connector (or to the trial stimulator if the operating room cable is hardwired thereto). The lead connector 452 is disposed along the first end portion 454 of the operating room cable 450 and the connector contacts within the lead connector are coupleable to the terminals 434 of the lead 403 (or lead extension). The trial stimulator connector 460 is disposed along the second end portion 456 of the operating room cable 450 and is coupleable to a stimulator connector 449 of the trial stimulator 448, either directly or via one or more operating room cable extensions. Any suitable terminals can be used in the operating room cable including rings, c-shaped contacts, plate contacts, pogo pins, and the like. Examples of terminals can be found in, for example, U.S. Pat. Nos. 7,539,542; 8,849,396; 9,101,775; 9,662,506; 10,130,806; 10,195,446; 10,639,485; and 11,045,656, all of which are incorporated herein by reference. In at least some embodiments, the trial stimulator connector 460 can be any suitable connector such as, for example, a USB-A, USB-B, USB-C, Micro USB, D-sub, or any other known connector or the like or any combination thereof. It will be understood that, in at least some embodiments, the operating room cable 450 can be hardwired to the trial stimulator 448.

At least some conventional operating room cables include loading the lead into a lead connector and then moving connector contacts into engagement with the lead and terminals on the lead. Such an arrangement often includes a flexible cable or flex circuit component to permit movement of the connector contacts within the body of the lead connector of the operating room cable. Such an arrangement can suffer from quality or cost issues.

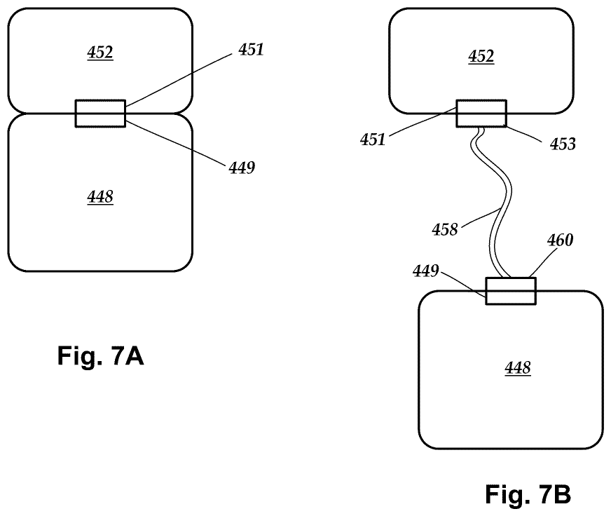

As described herein, an operating room cable includes a lead connector that receives the proximal end portion(s) of one or more leads or lead extensions. (It will be understood that any mention of the proximal end portion of a lead also includes the proximal end portion of a lead extension unless indicated otherwise.) These proximal end portion(s) are moved into or out of engagement with connector contacts of the lead connector. FIGS. 5A to 5C illustrate one embodiment of a lead connector 452 of an operating room cable 450 (FIG. 4) and FIGS. 6A to 6E illustrate another embodiment of the lead connector 452. It will be understood that the lead connector 452 can be part of an operating room cable, as illustrated in FIG. 4, or can be a separate device that can be coupled by an operating room cable to the external trial stimulator by an operating room cable, as illustrated in FIG. 7B, or directly attached to the external trial stimulator, as illustrated in FIG. 7A, or any combination thereof.

Any suitable trial stimulator connector 460 (FIG. 4) and elongated body 458 including, but not limited to, those used for conventional or existing operating room cables can be used with the lead connectors 452 disclosed herein (e.g., the lead connectors of FIGS. 5A to 5C and 6A to 6E). The elongated body 458 include multiple conductors (e.g., wires or cables) extending within a non-conductive sheath or jacket. The trial stimulator connector can be any standard or non-standard connector with multiple contacts for connecting to a trial stimulator. Alternatively, the operating room cable 460 is permanently connected or connectable (e.g., hardwired) to the trial stimulator and does not include a trial stimulator connector.

The lead connector 452 includes a housing 470 configured and arranged to receive a proximal end portion of at least one lead 403 (only part of which is illustrated in FIGS. 5A, 5B, 6B, and 6D for clarity) or lead extension and to electrically couple terminals 410 (FIG. 4) of the lead(s) to connector contacts 480 (FIG. 5B) of the lead connector. The connector contacts 480 (FIG. 5B) are electrically coupled to the trial stimulator connector 460 (FIG. 4) or the trial stimulator 448 (FIG. 4) through the elongated body 458.

The lead connector 452 can be configured to receive one, two, three, four, or any other suitable number of leads 403 (or lead extensions). For example, he lead connector 452 of FIGS. 5A to 5C is configured to receive two leads 403. The lead connector 452 can be configured to receive leads with any suitable number of terminals 410 (FIG. 4) including, for example, one, two, three, four, five, six, seven, eight, nine, ten, eleven, twelve, thirteen, fourteen, fifteen, sixteen, twenty, twenty-four, twenty-eight, thirty-two, or any other suitable number of terminals. In at least some embodiments, the lead connector 462 includes the same number of connector contacts 480 as the number of terminals on the proximal end portion of the lead that is received.

In at least some embodiments, the housing 470 has a first end 470a with one or more lead apertures 474 for each receiving the proximal end portion of a lead 403 and an opposing second end 470b from which the elongated body 458 extends. Any other suitable arrangement of lead aperture(s) 474 in the housing 470 can be used including, for example, a first end 470a with one or more lead apertures 474 and a second end 470b with one or more lead apertures. The elongated body 458 can extend from any suitable portion of the housing 470.

In at least some embodiments, each lead aperture 474 has a shape that allows the lead to move from a loading position (with the lead disengaged from the connector contacts 480, as illustrated in FIG. 6A) to an engagement position (with the lead engaging the connector contacts 480, as illustrated in FIG. 6B). For example, in at least some embodiments, each lead aperture has an elliptical or rectangular (with or without rounded corners) shape or a shape with straight or curved opposing sides and curved ends, as illustrated in FIG. 5A or any other suitable shape.

The housing 470 also includes sides 470c with one or more of the sides having an optional stylet aperture 476 extending along a portion or an entire length of the side. The optional stylet aperture 470 allows a stylet 336 (FIG. 3), which is partially inserted in the lead 403, to be inserted into the lead connector 452 as the proximal end portion of the lead is inserted into the lead aperture 474. The optional stylet aperture 476 intersects the lead aperture 474, as illustrated in FIG. 5A.

The housing 470 has a top surface 471 (or any other suitable surface) upon which one or more buttons 472 reside. In at least some embodiments, the button(s) 472 reside in a depression 471a formed in the top surface 471 of the housing 470 as illustrated in FIG. 5A. In at least some embodiments, the top surface 471 (or other suitable surface) of the housing 470 defines a sliding aperture 473 for each button 472 so that the button can be moved along the sliding aperture 473. Any suitable button 472 can be used. FIGS. 5A and 6A illustrate two different button shapes.

The housing 470 and button(s) 472 can be formed from any suitable material including, for example, plastic materials such as silicone, polyurethane, polyetheretherketone, or the like or any combination thereof. In at least some embodiments, the housing 470 and button(s) 472 are formed from molded plastic. In at least some embodiments, the housing 470 and button(s) 472 are made solely of non-conductive materials. In at least some embodiments, any button 472 or portion of the housing 470 can be textured to facilitate handling or manipulation.

Turning to FIGS. 5B and 6A to 6C, within the housing 470 the lead connector includes connector contacts 480 and a bracket 484. In at least some embodiments, the connector contacts 480 are disposed on, or attached to, a printed circuit board 482 or other substrate. The connector contacts 480 are electrically coupled to conductors (not shown), such as wires, cables, traces, or the like or any combination thereof, that extend along a length of the elongated body 458 (FIG. 4) of the operating room cable 450 (FIG. 4) and are electrically coupled to the trial stimulator connector 460 (FIG. 4), when present. In the illustrated embodiment, the connector contacts 480 are “M” shaped pins, but any other contact or suitably shaped pin can be used. The connector contacts 480 can be made of any suitable conductive material.

The bracket 484 defines a lead passage 483 that receives the proximal end portion of the lead 403 (a portion of which is illustrated in FIGS. 5B, 6A, and 6B) inserted through the lead aperture 474 (FIG. 5A). One embodiment of a bracket 484 is illustrated in FIG. 6C. This bracket 484 includes upper flanges 486a and lower flanges 486b that alternating or are interleaved along the lead passage 483. The upper flanges 486a and lower flanges 486b are attached to a bracket base 488. In at least some embodiments, the alternating or interleaving of the upper and lower flanges 486a, 486b permits molding the bracket 484 as a single piece.

The upper flanges 486a and lower flanges 486b, in combination, define the lead passage 483. The upper flanges 486a prevent or restrict upward movement of the lead 403 within the lead passage 483 and the lower flanges 486b prevent or restrict downward movement of the lead 403. In at least some embodiments, each of the upper flanges 486a are spaced apart longitudinally (e.g., along the lead passage 483) from adjacent lower flange(s) 486b to permit insertion of a connector contact 480 between adjacent upper and lower flanges, as illustrated, for example, in FIGS. 5B and 6B. In at least some embodiments, each of the upper and lower flanges 486a, 486b has a lip 481 (FIG. 6C) that prevents or restricts lateral removal of the lead 403 from the lead passage 483. In at least some embodiments, each of the upper and lower flanges 486a, 486b has a “J”-shape, as illustrated in FIGS. 5B and 6B, or a “U”-shape to provide the lip 481 (FIG. 6C).

In at least some embodiments, the upper and lower flanges 486a, 486b, in combination, of the bracket 484 have a vertical height, h, measured from a top surface of an upper flange 486a to a bottom surface of a lower flange 486b, as illustrated in FIG. 6C. In addition, the upper flanges 486a have a vertical height, hᵤ, of the lip 481 of the upper flanges and the lower flanges 486b have a vertical height, h₁, of the lip 481 of the lower flanges, as illustrated in FIG. 6C. In at least some embodiments, h>hᵤ+hₗ to leave a gap 485 between the upper flanges 486a and the lower flanges 486b. The gap 485 has a vertical height, h_{g}, where h_{g}=h−hᵤ−hₗ. In at least some embodiments, the gap 485 can facilitate molding the bracket 488 as a single piece.

In at least some embodiments, h_{g} is less than a diameter of the proximal end portion of the lead 403 so that the lead cannot be removed (or is difficult to remove) from the lead passage 483 through the gap 485. In at least some embodiments, h_{g} is larger than a diameter of the stylet shaft 377 (FIG. 3) so that the stylet shaft, when inserted through the optional stylet aperture (FIG. 5A), can pass between the upper and lower flanges 486a, 486b. In operation, a stylet 336 (FIG. 3) inserted into a lead 403 can be partially pulled out of the lead to expose a length of the stylet shaft 337. When the proximal end portion of the lead 403 is to be inserted into the lead aperture 474 of the lead connector 452, the stylet shaft 337 can be inserted (e.g., side-loaded) through the stylet aperture 476.

In at least some embodiments, the bracket 484 includes an end stop 490 at an end of the lead passage 483 to prevent insertion of the proximal tip of the lead 403 beyond the end stop. The end stop 490 can facilitate alignment of the terminals 410 of the lead with the connector contacts 480, as illustrated in FIG. 6B. In operation, a user inserts the proximal end portion of the lead 403 into the lead aperture 474 and continues to push the proximal end portion of the lead along the lead passage 483 until the proximal tip of the lead engages the end stop 490.

The bracket 488 is operatively coupled to a slide frame 492, which is attached to the button 472 as illustrated in FIGS. 5C, 6D, and 6E. The slide frame 492 includes one or more slots 494 and the bracket 488 includes a corresponding one or more pins 496 that fit into the slots. The slots 494 are slanted or angled and can be straight (FIGS. 6D and 6E) or curved (FIG. 5C). It will be understood that in other embodiments, the slots 494 can be defined in the bracket 488 and the pins 496 be part of the slide frame 492 or, in yet other embodiments, the slots 494 are distributed between the bracket 488 and the slide frame 492 with corresponding pins 496 on the bracket 488 and slide frame 492.

In operation, pushing the button 472 along the sliding aperture 473 moves the slide frame 492 so that the pins 494 of the bracket 488 move along the slots 494 of the slide frame 492 to raise or lower the bracket 488, as illustrated in FIGS. 6D and 6E, which correspond to FIGS. 6A and 6B, respectively. For the embodiments illustrated in FIGS. 5A to 6E, the bracket 488, when raised (for example, FIGS. 6A and 6D), is in a loading position allowing loading of a proximal end portion of a lead into the lead aperture 474. The bracket 488, when lowered (for example, FIGS. 5B, 5C, 6B, and 6E), is in an engagement position with the terminals 410 of the lead 403 engaging the connector contacts 480 to electrically couple the lead 403 to the external trial stimulator 448, when the operating room cable is coupled to the external trial stimulator.

In at least some embodiments, a bracket 488, a button 472, a sliding aperture 473, and a slide frame 492 are provided for each lead that is to be received by the lead connector 452, as shown in the illustrated embodiments. These embodiments have independent control of the receiving and engagement of each lead. In other embodiments, the button 472, sliding aperture 473, and slide frame 492 provided for two or more (or all) of the lead are the same so that moving from the loading position to the engagement position occurs simultaneously for all of these leads.

It will be understood that any directional terms used in this description are selected based on the orientation of the lead connector 452 and other components in the accompanying Figures. It will be understood that these directional terms are to be modified for lead connectors 452 in other orientations. For example, the terms “upper” and “lower” are interchanged if the lead connector 452 in the Figures is turned upside down.

FIG. 7A illustrates another arrangement of the lead connector 452 and external trial stimulator 448. In at least some embodiments of this arrangement, the lead connector 452 is directly attached or attachable to the external trial stimulator 448. In at least some embodiments, the lead connector 452 is permanently attached to the external trial stimulator 448 by hardwiring to the external trial stimulator.

In at least some other embodiments, the lead connector 452 is permanently or removably attached to the external trial stimulator 448 using an external connector 451 of the lead connector and a stimulator connector 449 of the external trial stimulator. In the embodiments of the lead connector 452 with an external connector 451, the connector contacts 480 of the lead connector 452 are electrically coupled to the external connector 451 by conductors, such as wires, traces, cables, or the like or any combination thereof.

In at least some of the embodiments with removable attachment, the arrangement also includes an operating room cable 458 for temporarily electrically coupling the lead connector 452 to the external trial stimulator 448, as illustrated in FIG. 7B. In at least some embodiments, the operating room cable 458 includes a first connector 453 for coupling to the external connector 451 of the lead connector 452 and a trial stimulator connector 460 for coupling to the stimulation connector 449 of the external trial stimulator 448.

As an example of use, during implantation, the lead connector 452 resides within the surgical, sterile field for coupling to the lead 403 extending from the patient and the operating room cable 458 extends from the surgical, sterile field to couple to the external trial stimulator 448 that is outside the surgical, sterile field. In at least some embodiments, after the implantation operation is completed, the operating room cable 458 is optionally removed and the lead connector 452 can be directly attached to the external trial stimulator 448.

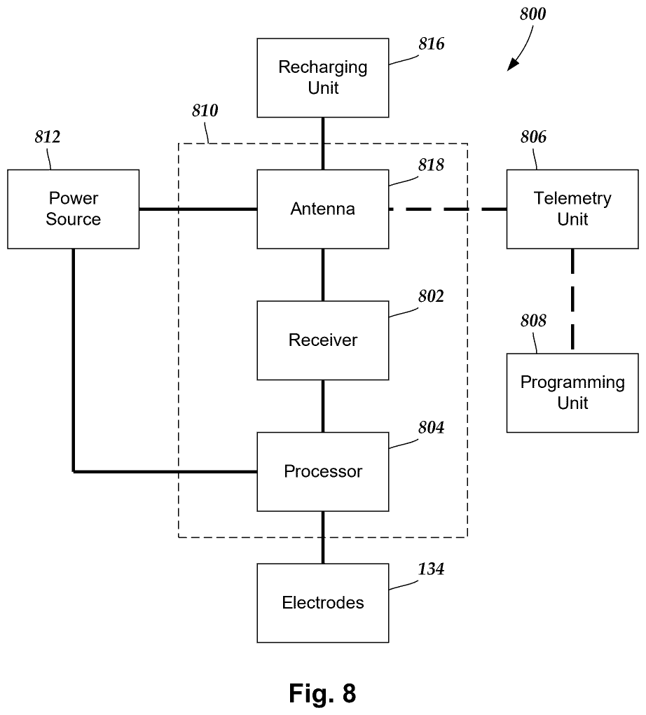

FIG. 8 is a schematic overview of one embodiment of components of an electrical stimulation system 800 including an electronic subassembly 810 disposed within a control module or trial stimulator. It will be understood that the electrical stimulation system can include more, fewer, or different components and can have a variety of different configurations including those configurations disclosed in the stimulator references cited herein.

Some of the components (for example, a power source 812, an antenna 818, a receiver 802, and a processor 804) of the electrical stimulation system can be positioned on one or more circuit boards or similar carriers within a sealed housing of an implantable pulse generator or external trial stimulator, if desired. Any power source 812 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 8,437,193, incorporated herein by reference. In at least some embodiments, an external trial stimulator can be configured to be coupled to an electrical socket, such as a wall socket, or other external power source.

If the power source 812 is a rechargeable battery, the battery may be recharged using the optional antenna 818, if desired. Power can be provided to the battery for recharging by inductively coupling the battery through the antenna to a recharging unit 816 external to the user. Examples of such arrangements can be found in the references identified above.

In one embodiment, electrical current is emitted by the electrodes 134 on the lead body to stimulate nerve fibers, muscle fibers, or other body tissues near the electrical stimulation system. The processor 804 is generally included to control the timing and electrical characteristics of the electrical stimulation system. For example, the processor 804 can, if desired, control one or more of the timing, frequency, strength, duration, and waveform of the pulses. In addition, the processor 804 can select which electrodes can be used to provide stimulation, if desired. In some embodiments, the processor 804 selects which electrode(s) are cathodes and which electrode(s) are anodes. In some embodiments, the processor 804 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 808 that, for example, allows modification of pulse characteristics. In the illustrated embodiment, the processor 804 is coupled to an optional receiver 802 which, in turn, is coupled to the optional antenna 818. This allows the processor 804 to receive instructions from an external source to, for example, direct the pulse characteristics and the selection of electrodes, if desired. In some embodiments of an external trial stimulator, the external trial stimulator can include or be coupleable to any suitable input device and any suitable display for direct input from a user, such as a clinician.

In one embodiment, the antenna 818 is capable of receiving signals (e.g., RF signals) from an external telemetry unit 806 which is programmed by the programming unit 808. The programming unit 808 can be external to, or part of, the telemetry unit 806. The telemetry unit 806 can be a device that is worn on the skin of the user or can be carried by the user and can have a form similar to a pager, cellular phone, or remote control, if desired. As another alternative, the telemetry unit 806 may not be worn or carried by the user but may only be available at a home station or at a clinician's office. The programming unit 808 can be any unit that can provide information to the telemetry unit 806 for transmission to the electrical stimulation system 800. The programming unit 808 can be part of the telemetry unit 806 or can provide signals or information to the telemetry unit 806 via a wireless or wired connection. One example of a suitable programming unit is a computer operated by the user or clinician to send signals to the telemetry unit 806.

The signals sent to the processor 804 via the antenna 818 and the receiver 802 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 800 to cease operation, to start operation, to start charging the battery, or to stop charging the battery. In other embodiments, the stimulation system does not include the antenna 818 or receiver 802 and the processor 804 operates as programmed.

Optionally, the electrical stimulation system 800 may include a transmitter (not shown) coupled to the processor 804 and the antenna 818 for transmitting signals back to the telemetry unit 806 or another unit capable of receiving the signals. For example, the electrical stimulation system 800 may transmit signals indicating whether the electrical stimulation system 800 is operating properly or not or indicating when the battery needs to be charged or the level of charge remaining in the battery. The processor 804 may also be capable of transmitting information about the pulse characteristics so that a user or clinician can determine or verify the characteristics.

The above specification provides a description of the manufacture and use of the invention. Since many embodiments of the invention can be made without departing from the spirit and scope of the invention, the invention also resides in the claims hereinafter appended.

What is claimed as new and desired to be protected is:

1. A device for electrically coupling a lead to an external trial stimulator, the device comprising:
a lead connector configured for electrically coupling the lead to the trial stimulator, the lead connector configured to receive a proximal end portion of the lead, the lead connector comprising
a housing defining a lead aperture in an end of the housing and a sliding aperture in a surface of the housing,
a plurality of connector contacts electrically coupleable to the external trial stimulator,
a slide frame,
a button coupled to the slide frame and configured to move along the sliding aperture, and
a bracket engaging the slide frame and defining a lead passage configured to receive the proximal end portion of the lead, the bracket comprising a plurality of upper flanges and a plurality of lower flanges, wherein the upper flanges and lower flanges alternate along the lead passage, wherein at least one of the slide frame or the bracket define at least one slanted slot and at least one of the slide frame or the bracket comprises at least one pin disposed within the at least one slanted slot of the other of the slide frame or the bracket,
wherein moving the button along the sliding aperture moves the bracket between a loading position, in which the proximal end portion of the lead is receivable through the lead aperture into the lead passage, and an engagement position, in which the proximal end portion of the lead, when received in the lead passage, engages the connector contacts.

2. The device of claim 1, wherein the lead connector is configured to receive the proximal end portions of a single lead or of a plurality of leads, wherein the lead connector comprises a plurality of the lead apertures and a plurality of the lead brackets, wherein each of the lead brackets defines a lead passage aligned with a different one of the lead apertures.

3. The device of claim 2 wherein the lead connector comprises a plurality of the buttons and a plurality of the slide frames, wherein each slide frame is coupled to a different one of the buttons and engages a different one of the brackets.

4. The device of claim 2, wherein the plurality of the lead brackets each engage the slide frame.

5. The device of claim 1, wherein the lead connector is configured for direct attachment to the external trial stimulator.

6. The device of claim 1, further comprising an elongated body having a first end and a second end, wherein the first end is coupled or coupleable to the external trial stimulator and the second end is coupled or coupleable to the lead connector.

7. The device of claim 1, wherein at least one of the at least one slanted slot defines a curved path.

8. The device of claim 1, wherein at least one of the at least one slanted slot defines a straight path.

9. The device of claim 1, wherein the housing further comprises a stylet aperture intersecting the lead aperture and extending along a side of the housing.

10. The device of claim 9, wherein the upper and lower flanges define a gap configured to allow a stylet extending from the lead and passing the stylet aperture to enter the lead passage.

11. The device of claim 1, wherein the bracket further comprises an end stop configured to prevent or resist further insertion of the proximal end portion of the electrical stimulation lead into the lead connector.

12. The device of claim 1, wherein each of the upper flanges and lower flanges has a “J” or “U” shape.

13. A trial stimulation system, comprising:
an external trial stimulator; and
the device of claim 1 coupleable, or coupled, to the external trial stimulator.

14. The trial stimulation system of claim 13, further comprising an elongated body coupled or coupleable to the lead connector and the external trial stimulator.

15. The trial stimulation system of claim 13, wherein the lead connector is configured for direct attachment to the external trial stimulator.

16. The trial stimulation system of claim 13, further comprising the lead coupleable to the lead connector.

17. The trial stimulation system of claim 16, further comprising a stylet configured for insertion into a stylet lumen of the lead.

18. The trial stimulation system of claim 16, further comprising a lead extension coupleable to the lead and the lead connector.

19. An insertion kit comprising:
the device of claim 1; and
at least one electrical stimulation lead, each electrical stimulation lead having a distal end portion and a proximal end portion and comprising
a plurality of electrodes disposed along the distal end portion of the electrical stimulation lead,
a plurality of terminals disposed along the proximal end portion of the electrical stimulation lead, and
a plurality of conductors coupling the electrodes to the terminals,
wherein the proximal end portion of the electrical stimulation lead is insertable into the lead connector.

20. A method for performing a trial stimulation on a patient, the method comprising:
providing the device of claim 1;
inserting a proximal end portion of the lead into the lead connector while the bracket is in the loading position; and
moving the bracket to the engagement position to engage terminals along the proximal end portion of the lead with the connector contacts of the lead connector.
